(19) United States
(12) Patent Application Publication
(10) Pub. No.: US 2020/0402425 A1
(43) Pub. Date: Dec. 24, 2020

(54) DEVICE FOR TRAINING USERS OF AN ULTRASOUND IMAGING DEVICE

(71) Applicant: Ariel-University Research and Development Company Ltd., Ariel (IL)

(72) Inventors: Ronnie TEPPER, Herzlia (IL); Nir SHVALB, Kibutz Bahan (IL); Boaz BEN-MOSHE, Herzliya (IL)

(73) Assignee: Ariel-University Research and Development Company Ltd., Ariel (IL)

(21) Appl. No.: 16/920,775

(22) Filed: Jul. 6, 2020

Related U.S. Application Data
(63) Continuation of application No. 14/387,548, filed on Sep. 24, 2014, now abandoned.
(60) Provisional application No. 61/618,791, filed on Apr. 1, 2012.

(51) Int. Cl. G09B 23/28; G06T 19/00; A61B 10/00

(52) U.S. Cl. CPC G09B 23/286; G06T 19/00; G06T 2219/008; G06T 2215/16; A61B 10/0048

(57) ABSTRACT

Disclosed are methods and devices for simulating ultrasound procedures and for training ultrasound users. Additionally disclosed are methods and devices for simulating needle insertion procedures, such as amniocentesis procedures, and for training physicians to perform such needle insertion procedures.

### RELATED APPLICATION

This application is a continuation of U.S. patent application Ser. No. 14/387,548 filed on Sep. 24, 2014, which is a National Phase of PCT Patent Application No. PCT/IB2013/052581 having International Filing Date of Mar. 31, 2013, which claims the benefit of priority of U.S. Provisional Patent Application No. 61/618,791 filed on Apr. 1, 2012. The contents of the above applications are all incorporated by reference as if fully set forth herein in their entirety.

### FIELD AND BACKGROUND OF THE INVENTION

The invention, in some embodiments, relates to the field of medical simulators, and more particularly, in some embodiments, to methods and devices for training ultrasound users for example, to perform medical sonography or needle-insertion procedures.

Ultrasound is a cyclic pressure wave with a frequency greater than about 20000 Hz, the upper limit of human hearing.

In sonography, such as medical sonography, ultrasound is used for imaging, especially of soft tissues. Medical sonography is used in many fields of medicine, including obstetrics, gynaecology, orthopaedics, neurology, cardiology, radiology, oncology, and gastroenterology.

A subtype of medical sonography, obstetric sonography is used to visualize an embryo or fetus in utero. Obstetric sonography is standard in prenatal care, and yields significant information regarding the health of the mother and fetus, as well as regarding the progress of the pregnancy. Obstetric sonography is used, for example, to determine the gender of the fetus, determine the gestational age, and detect fetal abnormalities, e.g., fetal organ anomalies or fetal developmental defects.

Obstetric sonography is also used during amniocentesis, helping to guide the amniocentesis needle to obtain a sample of the amniotic fluid without harming the fetus or the uterine wall.

Technicians and doctors are typically not trained to use obstetric sonography to detect fetal abnormalities. Thus, inexperienced doctors and technicians are typically incapable of identifying such abnormalities when these are encountered in practice.

Other subtypes of medical sonography are also used during invasive procedures, such as to image the soft tissue around a tumor or concretion being removed from the body in a laparoscopic surgery procedure.

In many fields, it is known to use training simulators. In sonography, training simulators typically comprise a physical mannequin. Such simulators are often insufficient because they fail to simulate motion of muscles during the procedure, or various types of abnormalities that can be encountered during the sonography.

For example, in obstetric sonography, training simulators comprise a physical mannequin of the belly of a pregnant woman including a physical model of a fetus. Such simulators are insufficient since the fetus model is static, and such training simulators fail to simulate an important factor of obstetric sonography, fetal movement. Further, in such training simulators, the maternal and embryo features are normal and therefore useless for training in identifying fetal abnormalities.

### SUMMARY OF THE INVENTION

The invention, in some embodiments, relates to the field of medical simulators, and more particularly, in some embodiments, to methods and devices for training ultrasound users to perform medical sonography, such as gynaecological sonography, cardiological sonography, gasteroenterological sonography, neurological sonography, musculoskeletal sonography, and CT scans, and to identify abnormalities potentially detected using such sonography methods.

According to an aspect of some embodiments of the invention there is provided an ultrasound simulator comprising:

a digital repository of virtual three-dimensional models, including at least one virtual three-dimensional model;

a processor associated with the repository and configured, during operation of the simulator to simulate, to use at least one of the virtual three-dimensional models in the repository;

a location-identifying surface associated with the processor; and

a physical ultrasound transducer simulator associated with the processor, the ultrasound transducer simulator comprising a three-dimensional orientation sensor configured to provide to the processor information regarding a three-dimensional orientation of the ultrasound transducer simulator relative to the location-identifying surface,

wherein at least one of the location-identifying surface and a device bearing the location identifying surface is operative to provide to the processor information regarding a two-dimensional location of the ultrasound transducer simulator on the surface.

In some embodiments, the ultrasound simulator also comprises a display associated with the processor, configured to visually display information to a user. In some such embodiments, the processor is operative to present on the display a section of one of the virtual three-dimensional models corresponding to the two-dimensional location and the three-dimensional orientation of the ultrasound transducer simulator relative to the surface.

In some embodiments, at least one of the three-dimensional models is a three-dimensional model of at least one three-dimensional geometrical shape, such as a sphere, an ellipsoid, a convex three dimensional geometrical shape, and a concave three-dimensional geometrical shape. In some embodiments, at least one of the three-dimensional models is a three-dimensional model of an irregular three-dimensional volume.

In some embodiments, at least one of the three-dimensional models is a three-dimensional anatomical model of at least a portion of an organism, in some embodiments the organism being a human.

In some embodiments, at least one of the three-dimensional models is a three-dimensional anatomical model of at least a portion of an embryo, in some embodiments a human embryo.

In some embodiments, at least one of the virtual three-dimensional models is a three-dimensional anatomical model of at least a portion of a fetus, in some embodiments a human fetus.

In some embodiments, at least one of the three-dimensional models is a three-dimensional anatomical model of at least a portion of a reproductive tract (e.g., uterus and/or fallopian tubes and/or ovaries), in some embodiments a human reproductive tract.

In some embodiments, at least one of the three-dimensional models is a three-dimensional anatomical model of at least a portion of a heart, in some embodiments a human heart.

In some embodiments, at least one of the three-dimensional models is a three-dimensional anatomical model of at least a portion of the circulatory system, in some embodiments a human kidney.

In some embodiments, at least one of the three-dimensional models is a three-dimensional anatomical model of at least a portion of a brain, in some embodiments a human brain.

In some embodiments, at least one of the three-dimensional models is a three-dimensional anatomical model of at least a portion of a digestive tract, in some embodiments a human digestive tract, for example, stomach, gall bladder or intestines.

In some embodiments, at least one of the three-dimensional models is a three-dimensional anatomical model of at least a portion of a muscle structure, in some embodiments a human muscle structure tract, for example, a limb including one or more of a muscle, a bone, a tendon and a joint.

In some embodiments, at least one of the three-dimensional models is an ultrasound model. In some such embodiments, the ultrasound model is constructed from multiple ultrasound images.

In some embodiments, at least one of the three-dimensional models is a Magnetic Resonance Imaging (MRI) model. In some such embodiments, the MRI model is constructed from multiple MRI images. In some such embodiments, the MRI model is modified to simulate the appearance of an ultrasound model.

In some embodiments, at least one of the three-dimensional models is an X-ray computed tomography (CT) model. In some such embodiments, the CT model is constructed from multiple CT images. In some such embodiments, the CT model is modified to simulate the appearance of an ultrasound model.

In some embodiments, the location-identifying surface comprises a touch sensitive surface, such as a touch pad or a touchscreen, for example a dedicated touchscreen, of a tablet computer or of a Smartphone. Typical suitable touchpad technologies include, but are not limited to, conductor matrix technology as described in U.S. Pat. No. 5,305,017 or capacitative shunt technology. Typical suitable touchscreen technologies include, but are not limited to, resistive, surface acoustic wave, capacitive, infrared grid, infrared acrylic projection, optical imaging, dispersive signal touch screens, and acoustic pulse recognition. In some such embodiments, the processor is the processor of the tablet computer or Smartphone bearing the touchscreen. In some such embodiments, the display is the display of the tablet computer or Smartphone, for example the display being overlaid on the touch-sensitive surface.

In some embodiments, the processor is a processor of a second electronic device separate from the location-identifying surface, such as a desktop computer, a laptop computer, a mobile phone, a Personal Digital Assistant (PDA), a tablet computer, or a smartphone. In some such embodiments, the display of the simulator is a display of the second electronic device separate from the location-identifying surface.

In some embodiments, the electronic device is configured for wired communication with the location-identifying surface. In some embodiments, the electronic device is configured for wireless communication with the location-identifying surface.

In some embodiments, the location-identifying surface is substantially similar to a computer mouse-pad.

In some embodiments, a device bearing the location-identifying surface comprises at least two cameras and an infra-red transmitter in order to identify the two-dimensional location. In some embodiments, the location-identifying surface comprises a magnetic sensor comprising a solenoid and a source of a magnetic field in order to identify the two-dimensional location. In some embodiments, the device bearing the location-identifying surface comprises a three-dimensional camera in order to identify the two-dimensional location.

In some embodiments, the ultrasound transducer simulator comprises a pressure sensor configured to measure the pressure applied by a user of the ultrasound transducer simulator on the location-identifying surface.

In some embodiments, the ultrasound transducer simulator comprises a tremor sensor configured to measure the hand tremors of a user of the ultrasound transducer simulator.

In some embodiments, the ultrasound transducer simulator is configured for wired communication with the processor. In some embodiments, the ultrasound transducer simulator is configured to have a wired connection to an electronic device including the processor to provide such wired communication.

In some embodiments, the ultrasound transducer simulator is configured for wireless communication with the processor.

In some embodiments the three-dimensional orientation sensor of the ultrasound transducer simulator includes a gyroscope, a compass, and an accelerometer, wherein the outputs of the gyroscope, compass and accelerometer are combined to identify the three-dimensional orientation of the ultrasound transducer simulator. Such components are commercially available and well-known in the field of gaming and mobile telephony.

In some embodiments, the three-dimensional orientation sensor of the ultrasound transducer simulator comprises a no-drift gyroscope. In some embodiments, the three-dimensional orientation sensor comprises three non-parallel solenoids, and a source of a magnetic field, wherein the three-dimensional orientation of the physical transducer simulator is calculated based on the percentage of current passing through each of the three solenoids. In some such embodiments, the three solenoids are mutually perpendicular. In some embodiments, the three-dimensional orientation sensor comprises a three-dimensional camera. In some embodiments, ultrasound transducer simulator comprises an encoder, such as a joystick, which is operative to indicate its three dimensional orientation.

In some embodiments, the three-dimensional orientation of the physical transducer simulator includes an indication of the yaw, pitch, and roll of the physical transducer simulator.

In some embodiments, the location-identifying surface and/or the device bearing the location-identifying surface is operative to provide to the processor information regarding a height of the ultrasound transducer simulator above the surface when there is no physical contact between the ultrasound transducer simulator and the surface.

In some embodiments the ultrasound simulator also includes a user-assessment module operative to assess at least one criterion of the performance of a user operating the ultrasound transducer simulator. In some embodiments, the user-assessment module forms part of the processor.

In some embodiments, the user-assessment module is configured to instruct the user to reach a specified section of the at least one virtual three-dimensional model used by the processor.

In some embodiments the user-assessment module instructs the user by presenting an image of the specified section on the display. In some embodiments the user-assessment module instructs the user by providing a verbal description of the specified section on the display. In some embodiments the user-assessment module instructs the user by providing an auditory description of the specified section.

In some embodiments the at least one criterion of the performance of a user comprises a number of attempts the user made to reach the specified section. In some embodiments the at least one criterion comprises a number of hand motions the user made to reach the specified section. In some embodiments the at least one criterion comprises the amount of pressure the user applied to the location-identifying surface via the ultrasound transducer simulator when attempting to reach the specified section.

In some embodiments, the user-assessment module provides a grade to the user, the grade being based on the user's performance in the at least one criterion.

In some embodiments, the user-assessment module provides to the user, in real time, guidance for reaching the specified section. In some embodiments the guidance is provided audibly (e.g., higher or lower tones). In some embodiments the guidance is provided on the display. In some embodiments the guidance is provided in a display overlaid on the location-identifying surface. In some embodiments the guidance is provided tactilely, such as by vibrations of the ultrasound transducer simulator. In some such embodiments, the ultrasound transducer simulator includes a tactile signal generator, such as a small piezoelectric speaker as known in the art of cellular telephony, for generation of a tactile guidance signal.

In some embodiments, the user-assessment module provides to the user, in real time, guidance for using appropriate pressure when attempting to reach the specified section. In some embodiments the guidance is provided aurally (e.g., higher or lower tones). In some embodiments the guidance is provided on the display. In some embodiments the guidance is provided in a display overlaid on the location-identifying surface. In some embodiments the guidance is provided tactilely, such as by vibrations of the ultrasound transducer simulator. In some embodiments the guidance is provided tactilely, such as by vibrations of the ultrasound transducer simulator. In some such embodiments, the ultrasound transducer simulator includes a tactile signal generator, such as a small piezoelectric speaker as known in the art of cellular telephony, for generation of a tactile guidance signal.

In some embodiments, the processor is configured to virtually move the virtual three-dimensional model during user-assessment, thereby simulating muscular or fetal motion during an ultrasound procedure.

In some embodiments, the ultrasound simulator includes a physical needle simulator associated with the processor, in addition to and different from the ultrasound transducer simulator, the physical needle simulator comprising:

a three-dimensional orientation sensor configured to sense and provide to the processor the three-dimensional orientation of the needle simulator; and

an insertion depth sensor configured to sense and provide to the processor information regarding the simulated depth of insertion of the needle simulator.

In some embodiments, the physical needle simulator is configured to simulate an amniocentesis needle. In some embodiments, the physical needle simulator is configured to simulate a laparoscopic needle. In some embodiments, the physical needle simulator is configured to simulate a biopsy needle.

In some embodiments, the insertion depth simulator comprises a distance sensor. In some such embodiments, the insertion depth simulator comprises a computer mouse, mounted onto the three-dimensional orientation sensor. In some such embodiments, the insertion depth simulator comprises a potentiometer. In some such embodiments, the insertion depth simulator comprises a linear encoder. In some such embodiments, the insertion depth simulator comprises a laser distance sensor. In some such embodiments, the insertion depth simulator comprises an ultrasonic distance sensor.

In some embodiments, the insertion depth simulator comprises a three-dimensional camera.

In some embodiments, the insertion depth simulator comprises a pressure sensor.

In some embodiments, the user-assessment module is configured to train the user to virtually insert a needle into a first virtual volume while not contacting a second virtual volume.

In some embodiments, the user-assessment module is configured to provide a warning indication to the user when the user is close to virtually contacting the second volume with the virtual needle. In some embodiments, the warning indication comprises a visual indication. For example, the visual indication may be provided on the display, in a display overlaid on the location identifying surface, or as a flashing warning light, such as on the physical needle simulator. In some embodiments, the warning indication comprises and aural indication. In some embodiments the warning indication comprises a tactile indication. In some such embodiments, the physical needle simulator includes a tactile signal generator, such as a small piezoelectric speaker as known in the art of cellular telephony, for generation of a tactile warning indication.

In some embodiments, the user-assessment module is configured to provide a contact indication to the user when the needle has virtually contacted the second volume. In some embodiments, the contact indication comprises a visual indication. For example, the visual indication may be provided on the display, in a display overlaid on the location identifying surface, or as a flashing warning light, such as on the physical needle simulator. In some embodiments, the contact indication comprises and aural indication. In some embodiments the contact indication comprises a tactile indication. In some such embodiments, the physical needle simulator includes a tactile signal generator, such as a small piezoelectric speaker as known in the art of cellular telephony, for generation of a tactile contact indication.

In some embodiments, such as in a first training stage, the first virtual volume comprises a first three-dimensional virtual volume and the second volume comprises a second three-dimensional virtual volume located near to, within, or surrounding the first volume.

In some embodiments the first virtual volume simulates a uterine volume containing amniotic fluid and the second virtual volume simulates an embryo or fetus, and the user-assessment module is configured to train the user to perform an amniocentesis procedure without harming the embryo or fetus.

In some embodiments the first virtual volume simulates a tumor tissue and the second virtual volume simulates healthy tissue, and the user-assessment module is configured to train the user to perform a biopsy of the tumor tissue without harming the healthy tissue.

In some embodiments the first virtual volume simulates a tissue of unknown character and the second virtual volume simulates healthy tissue, and the user-assessment module is configured to train the user to perform a biopsy of the tissue of unknown character without harming the healthy tissue in order to perform cytology tests to identify the type of tissue of unknown character.

In some embodiments, the first virtual volume simulates an undesired substance, and the second virtual volume simulates body tissue. For example, the first virtual volume may simulate a gall stone, a kidney stone, a lipoma, or a ganglion cyst.

In some embodiments, the user-assessment module virtually changes the orientation of at least part of the three-dimensional model during the assessment of the user, for example thereby simulating movement of the model.

According to an aspect of some embodiments of the invention there is also provided a method for simulating use of ultrasound imaging, comprising:

providing a digital repository of virtual three-dimensional models, including at least one virtual three dimensional model;

associating at least one of the virtual three-dimensional models in the repository with a processor;

from a physical ultrasound transducer simulator comprising a three-dimensional orientation sensor, providing to the processor information regarding a three-dimensional orientation of the ultrasound transducer simulator relative to a location-identifying surface functionally associated with the processor; and

providing to the processor information regarding a two dimensional location of the ultrasound transducer simulator on the location-identifying surface.

In some embodiments, the method also comprises visually displaying information to a user on a display, typically associated with the processor. In some such embodiments, the displaying comprises displaying a section of one of the virtual three-dimensional models corresponding to the two-dimensional location and the three-dimensional orientation of the ultrasound transducer simulator relative to the surface.

In some embodiments, the providing a repository comprises providing at least one three-dimensional model of at least one three-dimensional geometrical shape, such as a sphere, an ellipsoid, a convex three-dimensional geometrical shape and a concave three-dimensional geometrical shape. In some embodiments, the providing a repository comprises providing at least one three-dimensional model of an irregular three-dimensional volume.

In some embodiments, the providing a repository comprises providing at least one three-dimensional anatomical model of at least a portion of an organism, in some embodiments the organism being a human. In some embodiments, the providing a repository comprises providing at least one three-dimensional anatomical model of at least a portion of an embryo, in some embodiments a human embryo.

In some embodiments, the providing a repository comprises providing at least one three-dimensional anatomical model of at least a portion of a fetus, in some embodiments a human fetus.

In some embodiments, the providing a repository comprises providing at least one three-dimensional anatomical model of at least a portion of a reproductive tract (e.g., uterus and/or fallopian tubes and/or ovaries), in some embodiments a human reproductive tract.

In some embodiments, the providing a repository comprises providing at least one three-dimensional anatomical model of at least a portion of a heart, in some embodiments a human heart.

In some embodiments, at least one of the three-dimensional models is a three-dimensional anatomical model of at least a portion of the circulatory system, in some embodiments a human kidney.

In some embodiments, the providing a repository comprises providing at least one three-dimensional anatomical model of at least a portion of a brain, in some embodiments a human brain.

In some embodiments, the providing a repository comprises providing at least one three-dimensional anatomical model of at least a portion of a digestive tract, in some embodiments a human digestive tract, for example, stomach, gall bladder or intestines.

In some embodiments, the providing a repository comprises providing at least one three-dimensional anatomical model of at least a portion of a muscle structure, in some embodiments a human muscle structure tract, for example, a limb including one or more of a muscle, a bone, a tendon and a joint.

In some embodiments, the providing a repository comprises providing at least one ultrasound model. In some such embodiments, the ultrasound model is constructed from multiple ultrasound images.

In some embodiments, the providing a repository comprises providing at least one Magnetic Resonance Imaging (MRI) model. In some such embodiments, the MRI model is constructed from multiple MRI images. In some such embodiments, the MRI model is modified to simulate the appearance of an ultrasound model.

In some embodiments, at least one of the three-dimensional models is an X-ray computed tomography (CT) model. In some such embodiments, the CT model is constructed from multiple CT images. In some such embodiments, the CT model is modified to simulate the appearance of an ultrasound model.

In some embodiments, the associating a location-identifying surface with the processor comprises associating a processor of an electronic device, separate from the location identifying surface, with the location identifying surface. In some such embodiments, the electronic device comprises a desktop computer, a laptop computer, a mobile phone, or a Personal Digital Assistant (PDA). In some such embodiments, the displaying comprises displaying information to the user on a display of the electronic device.

In some embodiments, the providing information regarding the two-dimensional orientation of the ultrasound simulator transducer comprises: with an optoelectronic sensor, periodically acquiring images, and using an image processor, comparing succeeding images and translating changes in the images to velocity and direction. In some embodiments, the providing information also comprises using a distance measurer to determine whether or not there is contact with a surface, and to indicate the two dimensional location of such contact.

In some embodiments, the providing information regarding the two-dimensional orientation of the ultrasound simulator transducer comprises providing information from at least two cameras and from an infra-red transmitter. In some embodiments, the providing information regarding the two-dimensional orientation of the ultrasound simulator transducer comprises providing information from a magnetic sensor comprising a solenoid and a source of a magnetic field. In some embodiments, the location-identifying surface comprises the providing information regarding the two-dimensional orientation of the ultrasound simulator transducer comprises providing information from a three-dimensional camera.

In some embodiments, the method also comprises: from the ultrasound transducer simulator, providing to the processor information regarding the pressure applied by a user of the ultrasound transducer simulator on the location-identifying surface.

In some embodiments, the method also comprises from the ultrasound transducer simulator, providing to the processor information regarding hand tremors of a user of the ultrasound transducer simulator, which may be used to assess the user.

In some embodiments, the providing information regarding the three-dimensional orientation sensor of the ultrasound transducer simulator comprises combining outputs of a gyroscope, a compass and an accelerometer included in the ultrasound transducer simulator to identify the three-dimensional orientation of the ultrasound transducer simulator.

In some embodiments, the providing information regarding the three-dimensional orientation sensor of the ultrasound transducer simulator comprises providing information from a no-drift gyroscope. In some embodiments, the providing information regarding the three-dimensional orientation sensor of the ultrasound transducer simulator comprises calculating a percentage of current, generated by a source of a magnetic field, passing through each of three non-parallel solenoids. In some such embodiments, the three solenoids are mutually perpendicular. In some embodiments, the providing information regarding the three-dimensional orientation sensor of the ultrasound transducer simulator comprises providing information from a three-dimensional camera. In some embodiments, the providing information regarding the three-dimensional orientation sensor of the ultrasound transducer simulator comprises providing information from an encoder, such as a joystick, which is operative to indicate its three dimensional orientation.

In some embodiments, the providing information regarding the three-dimensional orientation sensor of the ultrasound transducer simulator comprises providing an indication of the yaw, pitch, and roll of the physical transducer simulator.

In some embodiments the method also includes assessing at least one criterion of the performance of a user operating the ultrasound transducer simulator.

In some embodiments, the assessing comprises instructing the user to virtually reach a specified section of the at least one virtual three-dimensional model used by the processor.

In some embodiments the instructing comprises presenting an image of the specified section on a display. In some embodiments the instructing comprises providing a verbal description of the specified section on a display. In some embodiments the instructing comprises providing an auditory description of the specified section.

In some embodiments the at least one criterion of the performance of a user comprises a number of attempts the user made to reach the specified section. In some embodiments the at least one criterion comprises a number of hand motions the user made to reach the specified section. In some embodiments the at least one criterion comprises the amount of pressure the user applied to the location-identifying surface via the ultrasound transducer simulator when attempting to reach the specified section. In some embodiments, the at least one criterion comprises a level of hand tremors of the user's hand while reaching the specified section.

In some embodiments, the assessing comprises providing a grade to the user, the grade being based on the user's performance in the at least one criterion.

In some embodiments, the assessing comprises providing to the user, in real time, guidance for reaching the specified section. In some embodiments the providing guidance comprises providing the guidance audibly (e.g., higher or lower tones). In some embodiments the providing guidance comprises providing the guidance on the display. In some embodiments the providing guidance comprises providing the guidance in a display overlaid on the location-identifying surface. In some embodiments the providing guidance comprises providing the guidance tactilely, such as by vibrations of the ultrasound transducer simulator.

In some embodiments, the assessing comprises providing to the user, in real time, guidance for using appropriate pressure when attempting to reach the specified section. In some embodiments the providing guidance comprises providing the guidance audibly (e.g., higher or lower tones). In some embodiments the providing guidance comprises providing the guidance on the display. In some embodiments the providing guidance comprises providing the guidance in a display overlaid on the location-identifying surface. In some embodiments the providing guidance comprises providing the guidance tactilely, such as by vibrations of the ultrasound transducer simulator.

In some embodiments, the method also comprises using the processor, virtually moving the virtual three-dimensional model during the assessing, thereby simulating muscular or fetal motion during an ultrasound procedure.

In some embodiments, the method also comprises:

associating a physical needle simulator with the processor, in addition to and different from the ultrasound transducer simulator;

from a three-dimensional orientation sensor included in the physical needle simulator, providing to the processor information regarding the three-dimensional orientation of the needle simulator; and

from an insertion depth sensor configured included in the physical needle simulator, providing to the processor information regarding the simulated depth of insertion of the needle simulator.

In some embodiments, the assessing comprises using the physical needle simulator, training the user to insert a needle into a first virtual volume while not contacting a second virtual volume.

In some embodiments, the assessing comprises providing a warning indication to the user when the user is close to virtually contacting the second volume with the needle. In some embodiments, providing a warning indication comprises providing a visual indication. For example, the visual indication may be provided on the display, in a display overlaid on the location identifying surface, or as a flashing warning light, such as on the physical needle simulator. In some embodiments, the providing a warning indication comprises providing an audible indication. In some embodiments the providing a warning indication comprises providing a tactile indication.

In some embodiments, the assessing comprises providing a contact indication to the user when the needle has virtually contacted the second volume. In some embodiments, the providing a contact indication comprises providing a visual indication. For example, the visual indication may be provided on the display, in a display overlaid on the location identifying surface, or as a flashing warning light, such as on the physical needle simulator. In some embodiments, the providing a contact indication comprises providing an audible indication. In some embodiments the providing a contact indication comprises providing a tactile indication.

In some embodiments, such as in a first training stage, the first virtual volume comprises a first three-dimensional virtual volume and the second virtual volume comprises a second three-dimensional virtual volume located near to, within, or surrounding the first virtual volume.

In some embodiments the first virtual volume simulates a uterine volume containing amniotic fluid and the second virtual volume simulates an embryo or fetus, and the assessing comprises training the user to perform an amniocentesis procedure without harming the embryo or fetus.

In some embodiments the first virtual volume simulates a tumor tissue and the second virtual volume simulates healthy tissue, and the assessing comprises training the user to perform a biopsy of the tumor tissue without harming the healthy tissue.

In some embodiments the first virtual volume simulates a tissue of unknown character and the second virtual volume simulates healthy tissue, and the assessing comprises training the user to perform a biopsy of the tissue of unknown character without harming the healthy tissue in order to perform cytology tests to identify the type of tissue of unknown character.

In some embodiments, the first virtual volume simulates an undesired substance, and the second virtual volume simulates body tissue. For example, the first virtual volume may simulate a gall stone, a kidney stone, a lipoma, or a ganglion cyst.

In some embodiments, the method also comprises virtually changing the orientation of at least part of the three-dimensional model during the assessing, for example thereby simulating movement of the model.

### BRIEF DESCRIPTION OF THE SEVERAL VIEWS OF THE DRAWINGS

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

In the Figures:

FIG. 1 is a schematic depiction, in cross-section, of an embodiment of a device comprising hardware and software for creating an ultrasound model repository according to an embodiment of the teachings herein;

FIGS. 2A, 2B, and 2C are schematic depictions of an embodiment of an ultrasound simulator according to the teachings herein;

FIG. 3 is a schematic block diagram representation of the ultrasound simulator of FIGS. 2A-2C;

FIGS. 4A and 4B are schematic depictions of an embodiment of a needle simulator according to the teachings herein; and

FIG. 5 is a schematic depiction of a simulator according to the teachings herein, combining the ultrasound simulator of FIGS. 2A-2C and FIG. 3 and the needle simulator of FIGS. 4A and 4B.

### DESCRIPTION OF SPECIFIC EMBODIMENTS OF THE INVENTION

The invention, in some embodiments, relates to the field of medical simulators, and more particularly, in some embodiments, to methods and devices for training ultrasound users to perform medical sonography, such as gynaecological sonography, cardiological sonography, gasteroenterological sonography, neurological sonography, musculoskeletal sonography, and CT scans, and to identify abnormalities seen in such tests.

As discussed above, methods and devices are needed in order to train users such as doctors and ultrasound technicians to recognize abnormalities and anomalies, such as embryonic abnormalities, or to safely guide medical devices, such as amniocentesis needles, using ultrasound imaging.

The principles, uses and implementations of the teachings of the invention may be better understood with reference to the accompanying description and figures. Upon perusal of the description and figures present herein, one skilled in the art is able to implement the teachings of the invention without undue effort or experimentation.

Before explaining at least one embodiment of the invention in detail, it is to be understood that the invention is not necessarily limited in its application to the details of construction and the arrangement of the components and/or methods set forth herein. The invention is capable of other embodiments or of being practiced or carried out in various ways. The phraseology and terminology employed herein are for descriptive purpose and should not be regarded as limiting.

According to an aspect of some embodiments of the invention there is provided an ultrasound simulator comprising:

a digital repository of virtual three-dimensional models, including at least one virtual three-dimensional model;

a processor associated with the repository and configured, during operation of the simulator to simulate, to use at least one of the virtual three-dimensional models in the repository;

a location-identifying surface associated with the processor; and

a physical ultrasound transducer simulator associated with the processor, the ultrasound transducer simulator comprising a three-dimensional orientation sensor configured to provide to the processor information regarding a three-dimensional orientation of the ultrasound transducer simulator relative to the location-identifying surface,

wherein at least one of the location-identifying surface and a device bearing the location-identifying surface is operative to provide to the processor information regarding a two-dimensional location of the ultrasound transducer simulator on the surface.

According to an aspect of some embodiments of the invention there is also provided a method for simulating the use of ultrasound imaging, comprising:

providing a digital repository of virtual three-dimensional models, including at least one virtual three-dimensional model;

associating at least one of the virtual three-dimensional models in the repository with a processor;

from a physical ultrasound transducer simulator comprising a three-dimensional orientation sensor, providing to the processor information regarding a three-dimensional orientation of the ultrasound transducer simulator relative to a location-identifying surface functionally associated with the processor; and

providing to the processor information regarding a two-dimensional location of the ultrasound transducer simulator on the surface.

In the context of the present application, the two dimensional location of the ultrasound transducer simulator on the surface is defined as a two dimensional point, or a two dimensional area, at which the ultrasound transducer simulator is in touching contact with the surface.

As used herein, when a numerical value is preceded by the either of the terms “about” and “around”, the terms “about” and “around” are intended to indicate +/−10%.

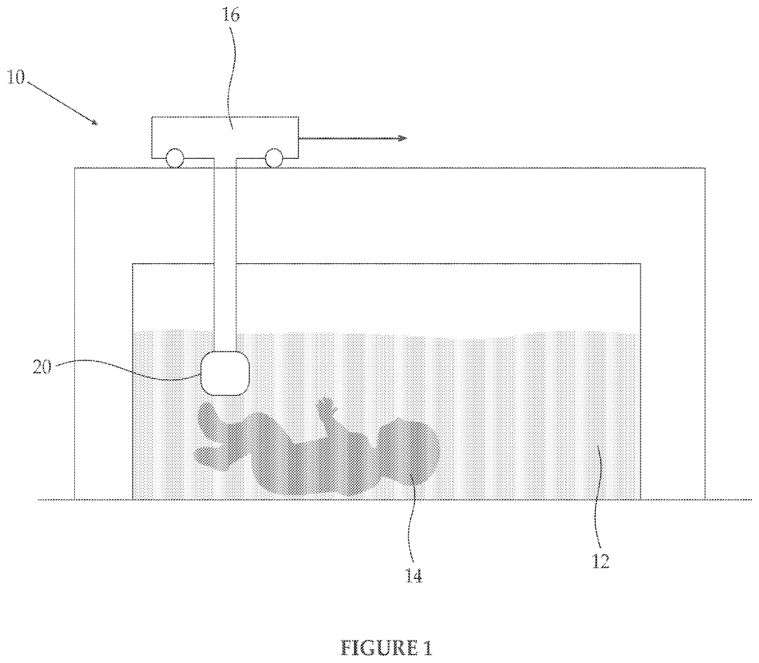

Reference is now made to FIG. 1, which is a schematic depiction, in cross-section, of an embodiment of a device 10 for creating an ultrasound model repository according to an embodiment of the teachings herein.

As seen in FIG. 1, a device 10 configured for obtaining sonographic images to be placed in an image repository includes a basin 12 which is filled with water, and in which is located an object 14 for imaging. In some embodiments, for example when creating a repository of gestational sonography images, the object 14 may comprise a deceased embryo. In some embodiments, for example when creating a repository of neurological sonography images, the object 14 may comprise a human brain. In some embodiments, for example when creating a repository of cardiological sonography images, the object 14 may comprise a human heart. It is appreciated that the object 14 may be any type of tissue, organ, body part or model thereof for which a repository of sonographic images is desired.

Above the basin 12 is located a robotic arm 16, which is movable along the X and Y axes of the basin 12. In some embodiments the robotic arm moves at a relatively slow speed, such as around 1 mm per second. At a bottom end of the robotic arm 16 is placed an ultrasound transducer 20, which is immersed in the water located in basin 12. Typically, the ultrasound transducer 20 is functionally associated with an ultrasound imaging device (not depicted), in some embodiments together configured to repeatedly acquire an ultrasound image of a plane.

For use for creating a repository of virtual three-dimensional images, the robotic arm 16 travels along the X and Y axes in the basin 12 while ultrasound transducer 20 is operational, such that the ultrasound transducer 20 obtains image information for multiple sections of the object 14. In some embodiments, the robotic arm 16 travels at a rate that allows transducer 20 to obtain approximately 300-400 section images per 15 to 20 centimeter of object 14. Once the section images are obtained, a processor (not shown) (e.g., of an associated ultrasound imaging device or of a different device) uses the section images to recreate a virtual three-dimensional model of the object 14, as known in the art of tomography for storage in a repository.

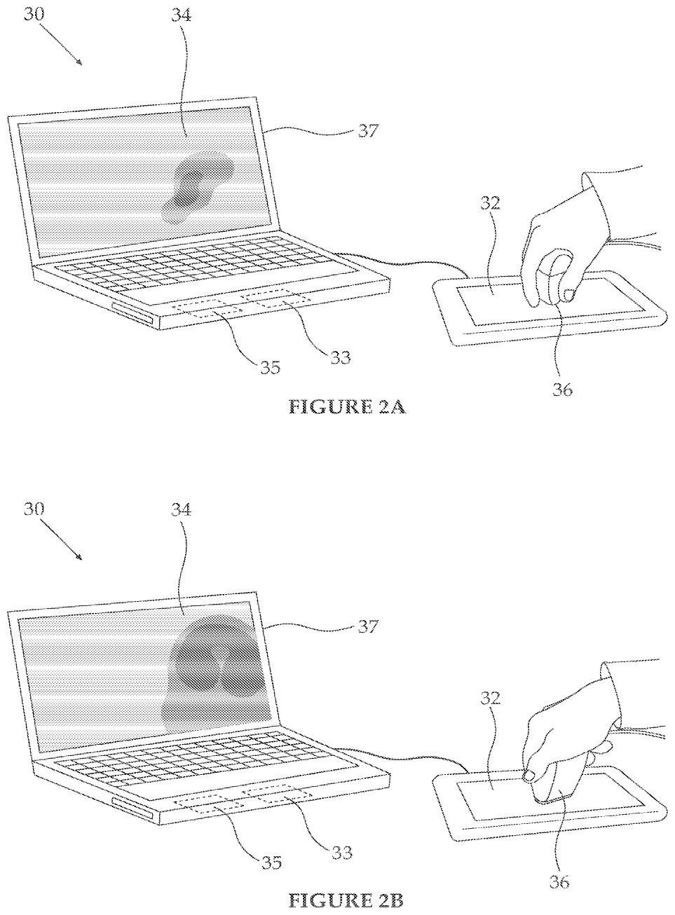

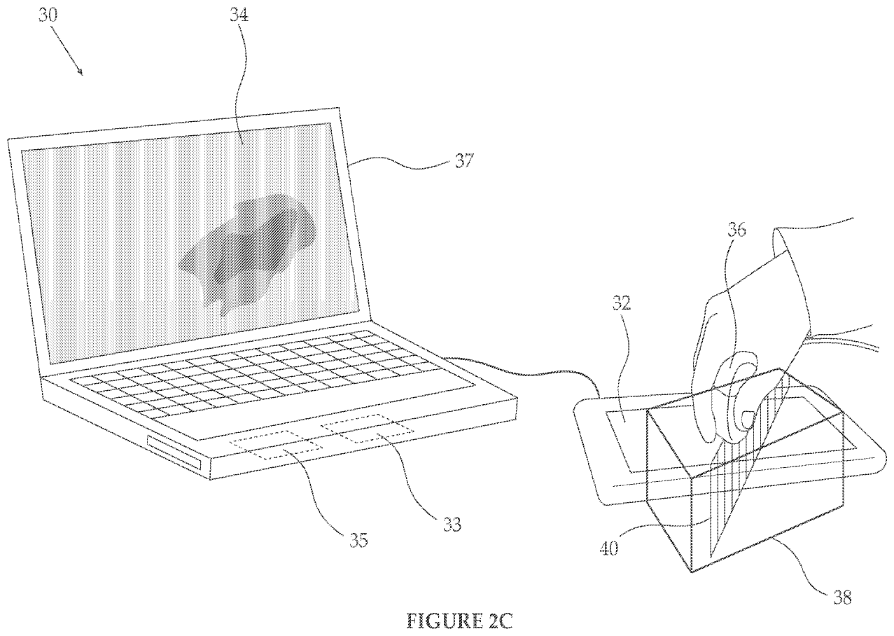

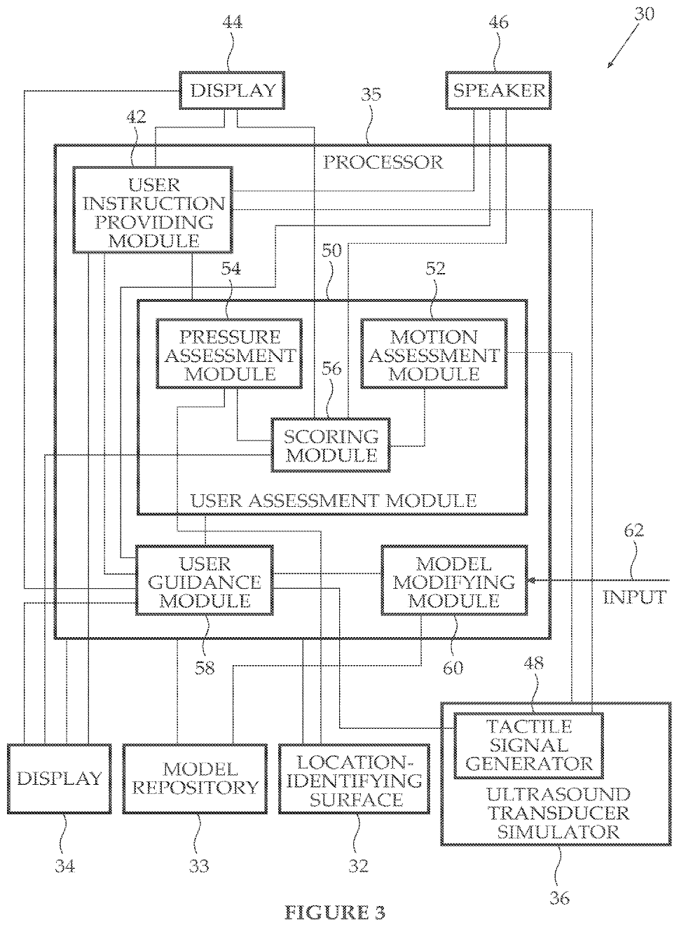

The three-dimensional model of the object created by the device 10 is added to an image repository (not shown), that can be used to implement the teachings herein, for example, together with an ultrasound simulator according to the teachings herein, an embodiment of which is described hereinbelow with reference to FIGS. 2A-2C and 3.

It is appreciated that the embodiment of FIG. 1 is an example only, and that other methods may be used for generating and/or populating an image repository cooperating with an ultrasound simulator as described hereinbelow with reference to FIGS. 2A-2C and 3. An image repository in accordance with the teachings herein may include any suitable type of models or images, such as for example Magnetic Resonance Imaging (MRI) images, Computerized Tomography (CT) images, sonography images, Computer Generated Images (CGI), and any three-dimensional models created therefrom. As such, any suitable method for obtaining such models or images is considered to be in the scope of the teachings herein.

It is further appreciated that an image and/or virtual model repository according to the teachings herein may include models and/or images of any volume, including three-dimensional geometrical volumes such as spheres, ellipsoids, convex three-dimensional volumes, concave three-dimensional volumes, irregular three-dimensional volumes, and three-dimensional volumes representing anatomical volumes, for example human or mammalian organs.

Reference is now made to FIGS. 2A, 2B, and 2C, which are schematic depictions of an embodiment of an ultrasound simulator according to the teachings herein, and to FIG. 3, which is a schematic block diagram representation of the ultrasound simulator of FIGS. 2A-2C.

As seen in FIGS. 2A-2C and in FIG. 3, an ultrasound simulator 30 includes a location-identifying surface 32, which simulates a body surface along which an ultrasound transducer simulator is moved. The location-identifying surface 32 is associated with a physical ultrasound transducer simulator 36, a processor 35, a three-dimensional model repository 33, including models, for instance as acquired in accordance with the discussed with reference to FIG. 1, and a display 34 configured to display to an user a simulated ultrasound image.

In some embodiments, the location-identifying surface 32 comprises a touch-sensitive surface, such that the touch sensitive surface provides to the processor 35 information regarding the two-dimensional location at which the physical transducer simulator 36 is positioned. The touch-sensitive surface may be any suitable touch-sensitive surface such as a touch screen known own in the art of user-machine interfaces. In some embodiments the touch-sensitive surface is of a tablet computer or smartphone, such as an iPad® or iPod® respectively, both commercially-available from Apple® Inc of Cupertino, Calif., USA. In some such embodiments, the processor 35 is the processor of the tablet computer/smartphone. In some embodiments, the touch sensitive surface comprises a touch pad, such as typically available in laptop computers, using a suitable technology. Suitable touchpads are commercially available, for example T650 by Logitech SA, Morges, Switzerland.

In some embodiments, the location-identifying surface 32 uses an optoelectronic sensor (e.g., as used in computer mouse technology) in order to identify the two-dimensional location at which the physical transducer simulator 36 is positioned.

In some embodiments, the simulator 30 uses multiple cameras and an infra-red transmitter associated with the physical ultrasound transducer simulator 36 to determine the two-dimensional location of the transducer simulator 36 relative to the location-identifying surface 32, in a technology similar to that provided by IntelliPen©.

In some embodiments, the simulator 30 uses a three-dimensional camera, such as a 3D Time of Flight camera commercially available from Mesa Imaging AG of Zurich, Switzerland, associated with the physical ultrasound transducer simulator 36 to determine the two-dimensional location of the transducer simulator 36 relative to the location-identifying surface 32.

In some embodiments, the location-identifying surface 32 uses a magnetic sensor comprising a solenoid and a magnetic field (e.g., generated by a magnetic-field generating component) in order to identify the two-dimensional location. In this case, the solenoid is located in the physical transducer simulator 36, and the two-dimensional location of the physical transducer simulator 36 is identified based on the magnitude of current passing through the solenoid.

In some embodiments, such as the embodiments depicted in FIGS. 2A-2C, the location-identifying surface 32 is separate from an electronic device 37 housing the processor 35, such as a desktop computer, a laptop computer, a smartphone, a mobile phone, a or Personal Digital Assistant (PDA). In some such embodiments, the display 34 is a display of the electronic device 37.

In some embodiments, such as the embodiment illustrated in FIGS. 2A-2C, electronic device 37 has a wired communication connection with the location-identifying surface 32. In some embodiments, electronic device 37 is configured for wireless communication with location-identifying surface 32 using any suitable wireless communication protocol, such as WiFi, Bluetooth®, and wireless telephony protocols such as GSM.

In some embodiments, the simulator 30 uses a three-dimensional camera, such as a 3D Time of Flight camera commercially available from Mesa Imaging AG of Zurich, Switzerland, associated with the physical ultrasound transducer simulator 36 to determine the two-dimensional location of the transducer simulator 36 relative to the location-identifying surface 32.

In some embodiments, the physical transducer simulator 36 is functionally associated with the processor 35, and provides the processor 35 information regarding its own three-dimensional orientation, including the yaw, pitch, and roll of the physical transducer simulator 36. In some embodiments, such as the embodiment illustrated in FIGS. 2A-2C, the physical transducer simulator 36 is connected to a device housing the processor 35, such as electronic device 37, by a wired communication connection. In some embodiments, the device housing the processor 35, such as electronic device 37, is configured for wireless communication with the physical transducer simulator 36 using any suitable wireless communication protocol, such as WiFi, Bluetooth®, and wireless telephony protocols such as GSM.

In some embodiments, the physical transducer simulator 36 comprises a gyroscope (not shown) used to identify the angular velocity of the transducer simulator 36, or, if the transducer simulator 36 is not moving, the three-dimensional orientation of the transducer simulator. The transducer simulator 36 may further include a compass (not shown) which indicates the direction in which the transducer simulator 36 is oriented and an accelerometer (not shown) used to obtain the direction in which the transducer simulator 36 is moving, or, when the transducer simulator 36 is not moving, the three-dimensional orientation of the transducer simulator 36. The three-dimensional orientation of the physical transducer simulator 36 is obtained by combining the information from the gyroscope, compass, and accelerometer using any suitable filter, such as a Kalman filter and/or LPF filters and/or HPF filters according to any method and using any suitable component with which a person having ordinary skill in the art is familiar.

It is appreciated that the gyroscope and the accelerometer provide very similar, if not identical, information regarding the orientation of the transducer simulator 36. However, due to the relatively noisy output of typical accelerometers, and to the drift problem often associated with gyroscopes, the combination of the outputs of the two provides more accurate positioning information than would be provided when using only one of the two. That said, in some embodiments a no-drift gyroscope is used, and obtain accurate positioning information for a transducer simulator 36.

Alternatively, in some embodiments transducer simulator 36 includes three non-parallel solenoids (e.g., mutually-orthogonally defining X, Y, and Z axes) and a source of a magnetic field in a specified plane. The current passing through each of the solenoids at any given moment is used to calculate the three-dimensional orientation of the transducer 36, in the usual way.

As a further alternative, in some embodiments physical transducer simulator 36 includes a mechanical device, similar to a joystick, which provides the three-dimensional orientation of the transducer simulator 36.

In some embodiments, the simulator 30 uses a three-dimensional camera, such as a 3D Time of Flight camera commercially available from Mesa Imaging AG of Zurich, Switzerland, associated with the physical ultrasound transducer simulator 36 to determine the three-dimensional orientation of the physical transducer simulator 36. This aspect is particularly useful when the three-dimensional camera is used also to identify the two dimensional location of the ultrasound simulator transducer 36 on surface 32.

During use of the simulator, for example for training, a specified virtual three-dimensional model from the repository is selected and uploaded to the processor 35. As seen in FIG. 2C, the orientation of the three-dimensional model is such that, if one were to enclose the specified virtual three dimensional model in a virtual box, indicated by reference numeral 38, one surface of the virtual box would lie against and, in some embodiments, would fill the location-identifying surface 32. It is appreciated that the exact virtual location and three-dimensional orientation of the three-dimensional model may be changed in real time or prior to the simulation, such as by an instructor, at random times or at regular time intervals.

The user places the physical transducer simulator 36 contacts the location-identifying surface 32 at a specific (two-dimensional location) with a three-dimensional orientation. The processor 35 is provided information regarding the two-dimensional location of the transducer 36 on the location-identifying surface 32, and the transducer simulator 36 provides the processor 35 information regarding its three-dimensional orientation relative to surface 32. In some embodiments, the processor 35 is provided information regarding the two-dimensional location of the transducer 36 on surface 32 directly from surface 32, for example when surface 32 is a touch surface operative to identify the two dimensional location at which it is contacted. In some embodiments, the processor 35 is provided information regarding the two-dimensional location of transducer 36 on surface 32 from a device associated with surface 32, such as a three dimensional camera operative to capture an image of transducer 36 located on surface 32.

In response, the processor 35 displays to the user on display 34 an image of a section of the selected three-dimensional virtual model, such that the section corresponds to an ultrasound image of the specified virtual three-dimensional model from the repository acquired by an ultrasound imaging transducer having the three-dimensional orientation of the transducer simulator 36 and at the location of the transducer simulator 36 relative to surface 32, as indicated by reference numeral 40 in FIG. 2C. As is evident from comparison of FIGS. 2A and 2B, a change in the two-dimensional location of transducer simulator 36 on surface 32 and/or in the three-dimensional orientation of transducer simulator 36 relative to surface 32 results in the display of an image of a different section of the model.

In some embodiments, the ultrasound simulator device 30 may be used for assessing the performance of a user. In some embodiments, as seen in FIG. 3, the processor 35 includes a user instruction providing module 42, which may be functionally associated with display 34, with an additional display 44 for presenting information to a user during the training or testing session, with speakers 46 for providing aural information and guidance to the user, or with a tactile signal generator 48 such as a small piezoelectric speaker as known in the art of cellular telephony, for generation of a tactile guidance signal for providing tactile information and guidance to the user. Tactile signal generator 48 typically is mounted on or otherwise attached to a hand-held ultrasound transducer simulator 36, such that it is contacted by the skin on a user of the transducer simulator 36 during operation thereof.

In some such embodiments, device 30 instructs the user to display an image of a specific section, for example by displaying an image or a verbal description of the specific section on display 34, on display 44, or overlaid on a surface 32, or by verbally specifying the section to be displayed, for example aurally using speakers 46.

In some such embodiments, device 30 is configured to assess whether the user has reached the correct section for display, how many attempts the user made until reaching the correct section, how many hand motions were required for the user to reach the correct section, and the amount of pressure applied by the user on surface 32. For this purpose processor 35 may include a user assessment module 50 including a motion assessment module 52 functionally associated with the ultrasound transducer simulator 36, a pressure assessment module 54 functionally associated with surface 32. The assessment information collected from modules 52 and 54 is summarized, and, in some embodiments, a scoring module 56 functionally associated with display 34, display 44, and/or speakers 46 presents the user with a grade of the test, and, in some cases, with comments and/or guidance for improvement, visually on display 34 and/or 44, and/or aurally using speakers 46.

In some embodiments, processor 35 also includes a user guidance module 58, functionally associated with the user assessment module 50 and configured, during a training or testing session, to guide the user to move the transducer simulator 36 (e.g., to the left or to the right), or to change the orientation of the transducer simulator 36, or to change the pressure applied to transducer simulator 36 in order to help the user reach the required section. In some such embodiments, the guidance information is provided as an overlay on the surface 32. In some such embodiments, the guidance information is provided to the user visually, such as on display 34 and/or on display 44. In some embodiments the guidance is provided audibly (e.g., higher or lower tones), for example using speakers 46. In some embodiments the guidance is provided tactilely, for example using tactile signal generator 48.

In some embodiments, processor 35 also includes a model modifying module 60 functionally associated with the repository 33, which is configured to modify (e.g., shape or orientation) of at least part of the virtual three-dimensional model during user-assessment, for example, to simulate muscular or fetal motion during an ultrasound procedure. The model modifying module 60 may modify the model at regular intervals, at random intervals, or upon receipt of input from an assessing entity as indicated by input arrow 62. In some embodiments, model modifying module 60 is functionally associated with the user assessment module 50 and specifically with user guidance module 58, so that guidance provided to the user of transducer simulator 36 may be updated upon modification by module 60 of the model being used for user assessment.

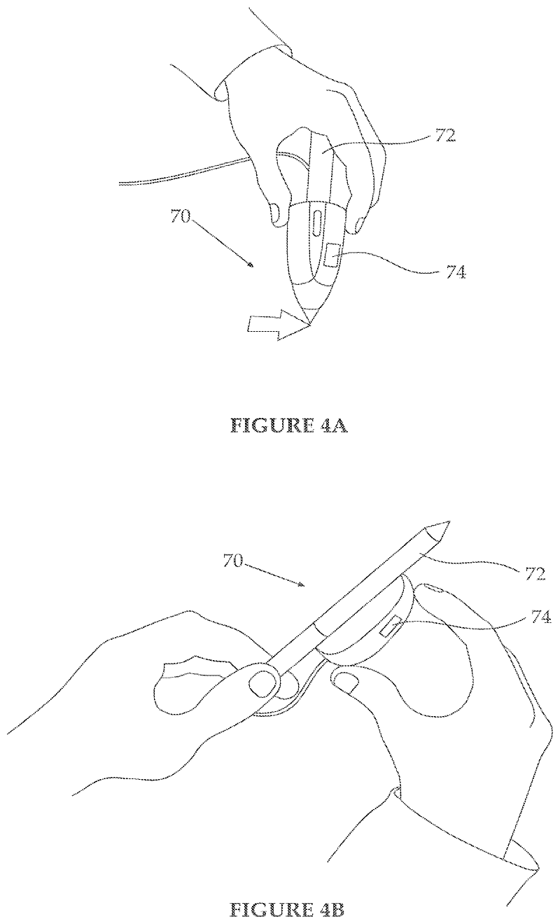

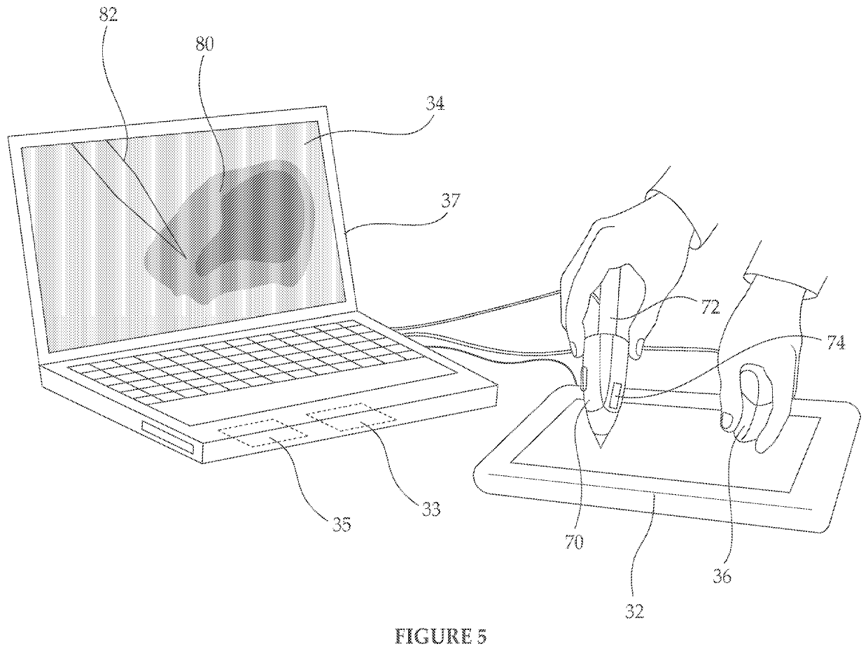

Reference is now made to FIGS. 4A and 4B, which are schematic depictions of an embodiment of a needle simulator and according to the teachings herein and to FIG. 5, which is a schematic depiction of a simulator and training device according to the teachings herein combining the ultrasound simulator and user training device of FIGS. 2A-2C and FIG. 3 and the needle simulator of FIGS. 4A and 4B.

As seen in FIGS. 4A to 5, a simulator and training device according to the teachings herein includes, in addition to the elements of device 30 described hereinabove with reference to FIGS. 2A-2C and FIG. 3, a physical needle simulator 70 associated with the processor 35. The needle simulator 70 includes a three-dimensional orientation sensor 72 configured to provide processor 35 with the orientation of the needle simulator 70 relative to surface 32, and a virtual insertion depth sensor 74 configured to provide processor 35 with a value indicative of a depth to which the needle simulator virtually penetrates into surface 32.

In some embodiments, the three-dimensional orientation sensor 72 comprises a pen associated with a tablet computer, such as the Intuos3 Grip Pen commercially available from Wacom Company Ltd. of Tokyo, Japan.

In some embodiments, the insertion depth simulator 74 comprises a component similar to a computer mouse, mounted onto the three-dimensional orientation sensor 72, such that the lower the device is along the three-dimensional orientation sensor 72 indicates deeper virtual insertion of the needle simulator. In some such embodiments, the mouse is associated with the processor and provides to the processor information regarding its height over the surface 32, thereby providing to the processor information regarding the virtual depth to which the needle simulator is inserted.

In some embodiments, the insertion depth simulator 74 comprises a distance sensor. In some such embodiments, the distance sensor comprises a potentiometer. In some such embodiments, the distance sensor comprises a linear encoder. In some such embodiments, the distance sensor comprises a laser distance sensor. In some such embodiments, the distance sensor comprises an ultrasonic distance sensor.

In some embodiments, the three-dimensional orientation sensor 72 and/or the insertion depth simulator 74 comprises a three-dimensional camera, such as a 3D Time of Flight camera, commercially available from Mesa Imaging AG of Zurich, Switzerland, which camera may provide information regarding the three-dimensional orientation of the simulated needle and/or information regarding the depth to which the needle was inserted.

In some such embodiments, the insertion depth simulator 74 comprises a pressure sensor.

In some embodiments, such as the embodiment illustrated in FIG. 4, electronic device 37 housing processor 35 has a wired communication connection with the needle simulator 70. In some embodiments, electronic device 37 is configured for wireless communication with needle simulator 70 using any suitable wireless communication protocol, such as WiFi, Bluetooth®, and wireless telephony protocols such as GSM.

In some embodiments, the physical needle simulator 70 is configured to simulate an amniocentesis needle. In some embodiments, the physical needle simulator 70 is configured to simulate a laparoscopic needle. In some embodiments, the physical needle simulator 70 is configured to simulate a biopsy needle.

In some embodiments, a physical needle simulator is configured to simulate a different type of hard device used to penetrate into a body and guided by a user to a location in the body with the help of ultrasound imaging.

In use, a virtual three-dimensional model from the model repository 33 is specified and uploaded by the processor 35, in a similar manner to that described hereinabove with reference to FIG. 2C.

In addition to placing the physical transducer simulator 36 on the location-identifying surface 32 as described hereinabove with reference to FIGS. 2A to 3, the user being trained to use a needle together with an ultrasound imaging transducer places the needle simulator 70 on the location-identifying surface 32.

The processor receives information regarding the two-dimensional location of the transducer simulator 36 and information regarding the transducer's three-dimensional of the transducer simulator 36, substantially as described above.

Additionally, the needle simulator 70 provides the processor 35 with information regarding the three-dimensional orientation of the needle simulator 70 and about the virtual depth of insertion of the needle simulator 70. In some embodiments, the information regarding the three-dimensional orientation is provided by the three-dimensional orientation sensor 72 and the information regarding the virtual depth of insertion of the needle is provided by the insertion depth sensor 74.

In response, the processor 35 provides to display 34 an image of a section of the model, indicated by reference numeral 80, such that the section corresponds to the three-dimensional orientation of the transducer 36, with a superimposed image 82 of a virtual needle having a location corresponding to the location, orientation and virtual insertion depth of the needle simulator 70.

As described hereinabove, in some embodiments the ultrasound simulator device 30 and the needle simulator 70 may be used for assessing the performance of a user, by instructing the user to insert the needle into a certain place in the three dimensional model and assessing the user's performance, substantially as described hereinabove with reference to FIGS. 2A-2C and 3.

In some embodiments, a user assessment module of processor 35, such as user assessment module 50 of FIG. 3, is configured to train the user to virtually insert a needle into a first virtual volume while not contacting a virtual second volume.

In some embodiments, such as in a first training stage, the first virtual volume comprises a first three-dimensional volume and the second virtual volume comprises a second three-dimensional volume located near to, within, or surrounding the first virtual volume.

In some embodiments the first virtual volume simulates a uterine volume with amniotic fluid and the second virtual volume simulates an embryo or fetus thereinside, and the user-assessment module is configured to train the user to perform an amniocentesis procedure without harming the embryo or fetus.

In some embodiments the first virtual volume simulates a tumor tissue and the second virtual volume simulates healthy tissue, and the user-assessment module is configured to train the user to perform a biopsy of the tumor tissue without harming the healthy tissue.

In some embodiments the first virtual volume simulates a tissue of unknown character and the second virtual volume simulates healthy tissue, and the user-assessment module is configured to train the user to perform a biopsy of the tissue of unknown character without harming the healthy tissue in order to perform cytology tests to identify the type of tissue of unknown character.

In some embodiments, the first virtual volume simulates an undesired substance, and the second virtual volume simulates body tissue. For example, the first virtual volume may simulate a gall stone, a kidney stone, a lipoma, or a ganglion cyst.

In some embodiments, the user-assessment module is configured to provide a warning indication to the user when the needle simulator position, orientation and virtual insertion depth correspond to a simulated needle being dangerously close to the second virtual volume. For example, the user may be warned if the simulated needle is within one millimeter of the second virtual volume.

In some embodiments, the warning indication comprises a visual indication. For example, the visual indication may be provided on the display, such as display 34 or 44 of FIG. 3, in a display overlaid on the location identifying surface 32, or as a flashing warning light (not shown), such as on the physical needle simulator. In some embodiments, the warning indication comprises an audible indication, provided for example using speakers, such as speakers 46 of FIG. 3. In some embodiments the warning indication comprises a tactile indication, which may be provided, for example, by a tactile signal generator (not shown) mounted onto the needle simulator 70. The tactile signal generator may be, for example, a small piezoelectric speaker as known in the art of cellular telephony.

In some embodiments, the user-assessment module is configured to provide a contact indication to the user when the needle simulator position, orientation and virtual insertion depth correspond to a simulated needle being in contact with the second virtual volume.

In some embodiments, the contact indication comprises a visual indication. For example, the visual indication may be provided on the display, such as display 34 or 44 of FIG. 3, in a display overlaid on the location identifying surface 32, or as a flashing contact light (not shown), such as on the physical needle simulator. In some embodiments, the contact indication comprises an audible indication, provided for example using speakers, such as speakers 46 of FIG. 3. In some embodiments the contact indication comprises a tactile indication, which may be provided, for example, by a tactile signal generator (not shown) mounted onto the needle simulator 70. The tactile signal generator may be, for example, a small piezoelectric speaker as known in the art of cellular telephony.

As described hereinabove with reference to FIG. 3, in some embodiments, at least part of the three-dimensional model may be changed, e.g. virtually rotated or moved during assessment of the user. In some embodiments, the processor 35 is configured to carry out such changes at random intervals or at regular intervals. In some embodiments, an assessor or training professional may change the virtual orientation of the virtual three-dimensional model during the needle insertion simulation by providing input to processor 35, substantially as described hereinabove with reference to FIG. 3, thereby simulating a change during the procedure, such as embryonic or muscular movement, and to train the user to avoid the simulated needle contacting and/or harming the second virtual volume even if the volume or a portion thereof moves. For example, in a simulation of amniocentesis, the supervisor may change the virtual orientation of at least a portion of the embryo or fetus, thereby simulating movement of a fetal limb.

As described hereinabove with reference to FIGS. 2A-2C and 3, in some embodiments, the user assessment module provides a score for user performance. In the case of needle insertion simulation, the score is based on the pressure applied to the ultrasound transducer simulator, the number of times the user had to try to perform the test, and/or on the distance of the simulated needle from the second volume of the three-dimensional model.

It is appreciated that certain features of the invention, which are, for clarity, described in the context of separate embodiments, may also be provided in combination in a single embodiment. Conversely, various features is of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. An ultrasound simulator comprising:
a digital repository, the repository including at least one virtual three-dimensional model of a torso, the model comprising a plurality of simulated body organs;
a processor associated with said repository and configured to use at least one of said virtual three-dimensional models in said repository;
a location-identifying surface associated with said processor, the location-identifying surface being a touch sensitive surface which is built in to a body simulating unit, the location-identifying surface further providing pressure sensing; and
a physical ultrasound transducer simulator associated with said processor, said ultrasound transducer simulator comprising an orientation sensor configured to provide to said processor a measurement of an orientation of said ultrasound transducer simulator relative to said location-identifying surface, said processor configured to relate said measurement to a surface location on said at least one three-dimensional model, different surface locations being in proximity to different ones of said simulated body organs, the simulator further being configured to provide an ultrasound image having the orientation of the physical ultrasound transducer simulator at said two-dimensional location on said location-identifying surface, the ultrasound image provided being a two-dimensional section through said three-dimensional model, the section changeably moving or rotating within said three-dimensional model as said ultrasound transducer simulator is moved or rotated, thereby to simulate ultrasound viewing of any one of said simulated organs.

2. The ultrasound simulator of claim 1, wherein at least one of said three-dimensional models is at least one of an ultrasound model, a Magnetic Resonance Imaging (MRI) model and a X-ray computed tomography (CT) model.

3. The ultrasound simulator of claim 1, also comprising a user-assessment module operative to assess at least one criterion of said performance of a user operating said ultrasound transducer simulator, and configured to instruct said user to reach a specified section of said at least one virtual three-dimensional model used by said processor.

4. The ultrasound simulator of claim 3, wherein said user-assessment module is also configured to provide a grade to said user, said grade being based on said user's performance in said at least one criterion.

5. The ultrasound simulator of claim 3, wherein said user-assessment module is configured to provide to said user, in real time, at least one of guidance for reaching said specified section and guidance for using appropriate pressure when attempting to reach said specified section.

6. The ultrasound simulator of claim 1, also comprising a physical needle simulator associated with said processor, in addition to and different from said ultrasound transducer simulator, said physical needle simulator comprising:
a three-dimensional orientation sensor configured to sense and to provide to said processor said three-dimensional orientation of said needle simulator; and
an insertion depth sensor configured to sense and to provide to said processor information regarding said simulated depth of insertion of said needle simulator.

7. The ultrasound simulator of claim 6, wherein said user assessment module is configured to train said user to insert a needle into a first volume while not contacting a second volume.

8. The ultrasound simulator of claim 6, wherein said user-assessment module is configured to at least one of:
provide a warning indication to said user when a position of said needle simulator and a virtual insertion depth of said needle simulator correspond to a simulated needle being within a predetermined distance of contacting said second volume; and
provide a contact indication to said user when a position of said needle simulator and a virtual insertion depth of said needle simulator correspond to a simulated needle contacting said second volume.

9. A method for simulating an ultrasound, comprising:
providing a digital repository, the repository including at least one virtual three-dimensional model of a torso, the model comprising a plurality of simulated organs located around said torso;
associating at least one of said virtual three-dimensional models in said repository with a processor;
from a physical ultrasound transducer simulator comprising a three-dimensional orientation sensor, independently providing to said processor an orientation of said ultrasound transducer simulator relative to a location-identifying surface, said location-identifying surface being a touch-sensitive surface;
providing pressure information from said touch-sensitive surface;
providing to said processor information regarding a two-dimensional location of said ultrasound transducer simulator on said touch-sensitive surface;
relating said two-dimensional location on said touch sensitive surface to a location on said associated three-dimensional model; and
providing in real time an ultrasound image having the three-dimensional orientation of the physical ultrasound transducer simulator at said two-dimensional location on said associated three-dimensional model, thereby to allow a user to find a required one of said simulated organs and obtain a simulated ultrasound image thereof.

10. The method of claim 9, wherein said providing a repository comprises providing at least one ultrasound model.

11. The method of claim 9, wherein said providing a repository comprises providing at least one of at least one Magnetic Resonance Imaging (MRI) model and at least one X-ray computed tomography (CT) model.

12. The method of claim 9, also comprising assessing at least one criterion of said performance of a user operating said ultrasound transducer simulator, wherein said assessing comprises instructing said user to reach a specified section of said at least one virtual three-dimensional model used by said processor.

13. The method of claim 12, wherein said assessing comprises providing a grade to said user, said grade being based on said user's performance in said at least one criterion.

14. The method of claim 12, wherein said assessing comprises providing to said user, in real time, at least one of guidance for reaching said specified section and guidance for using appropriate pressure when attempting to reach said specified section.

15. The method of claim 9, also comprising:
associating a physical needle simulator with said processor, in addition to and different from said ultrasound transducer simulator;
from a three-dimensional orientation sensor included in said physical needle simulator, providing to said processor information regarding said three-dimensional orientation of said needle simulator; and
from an insertion depth sensor configured included in said physical needle simulator, providing to said processor information regarding said simulated depth of insertion of said needle simulator.

16. The method of claim 13, wherein said assessing comprises using said physical needle simulator, training said user to insert a needle into a first volume while not contacting a second volume.

17. The method of claim 14, wherein said assessing comprises at least one of:
providing a warning indication to said user when a position of said needle simulator and a virtual insertion depth of said needle simulator correspond to a simulated needle being within a predetermined distance of contacting said second volume; and
providing a contact indication to said user when a position of said needle simulator and a virtual insertion depth of said needle simulator correspond to a simulated needle contacting said second volume.
